(19) United States
(12) Patent Application Publication
(10) Pub. No.: US 2020/0282192 A1
(43) Pub. Date: Sep. 10, 2020

(54) SUTURE-LESS CHEST TUBE

(71) Applicant: Texas Tech University System, Lubbock, TX (US)

(72) Inventor: Robert F. Stump, El Paso, TX (US)

(21) Appl. No.: 16/644,492

(22) PCT Filed: Sep. 18, 2018
(86) PCT No.: PCT/IB2018/057193

§ 371 Date: Mar. 4, 2020

Related U.S. Application Data
(60) Provisional application No. 62/559,738, filed on Sep. 18, 2017.

(51) Int. Cl. A61M 27/00; A61M 1/04

(52) U.S. Cl. CPC A61M 27/00; A61M 2025/0233; A61M 1/04

(57) ABSTRACT

A system, method and apparatus for performing a thoracostomy comprises a tube for insertion in a patient's chest cavity, an inflatable sac configured on a first section of the tube, an inflation valve for inflating the inflatable sac, and a flange associated with a second section of the tube configured for securing the tube wherein the first section of the tube is inserted into the patient's body and the second section of the tube is external to the patient's body.

### TECHNICAL FIELD

Embodiments are generally related to the field of medical devices. More particularly, the present embodiments are directed to chest tubes and chest tube thoracostomy. Embodiments are further related to improved methods and systems for chest tubes that do not require sutures.

### BACKGROUND

In emergency and trauma medicine, chest tubes are a common and critically important medical device. Typically, chest tubes are used to treat pneumothorax (air around the lung) and hemothorax (blood or fluid around the lung). These conditions are often the result of chest trauma, such as a gunshot or stab wound, and require immediate emergency medical care. Chest tubes are also used to treat other spontaneous conditions including pneumothorax or hemothorax due to cancer, COPD, and body habitus.

Current practice for inserting a chest tube includes making a skin incision across the rib, inserting the chest tube, and then using sutures to secure the tube. The procedure is invasive, painful, and risky. Once the chest tube is inserted, sutures are used to hold the tube in place, and a dressing might be applied. In practice, it is extremely uncommon for a truly air tight seal to be achieved in this way.

Prior art chest tubes, and the methods used to install them, can be time consuming, ineffective, and dangerous. For example, the suturing method is very time intensive to complete, which can result in catastrophic time delays in emergency situations. It is difficult to adequately secure the chest tube in place and the incision may be difficult to render sufficiently airtight. As a result, a cumbersome combination of bandages, dressings, and tape are often applied, one at a time, in an effort to seal the area where the tube is sutured to the skin. In addition, sharp objects necessary for sutures increase the transmission risk of infectious diseases to healthcare providers by needle stick injury.

Accordingly, there is a need in the art for improved chest tubes that are safer and less time consuming to properly install, as disclosed herein.

### BRIEF SUMMARY

The following summary is provided to facilitate an understanding of some of the innovative features unique to the embodiments disclosed and is not intended to be a full description. A full appreciation of the various aspects of the embodiments can be gained by taking the entire specification, claims, drawings, and abstract as a whole.

Various objects and advantages of this use will become apparent from the following description taken in conjunction with the accompanying drawings which describe certain embodiments of this invention.

It is, therefore, one aspect of the disclosed embodiments to provide novel methods, systems, and apparatuses associated with chest tubes.

Specifically, in an aspect of the embodiments, a suture-less chest tube that can be installed quickly, and with the use of fewer potentially infectious tools, is disclosed.

The aforementioned aspects and other objectives and advantages can now be achieved as described herein. For example, in certain embodiments a system comprises a tube for insertion in a patient's chest cavity, an inflatable sac configured on a first section of the tube, an inflation valve for inflating the inflatable sac, and a flange associated with a second section of the tube configured for securing the tube. The first section of the tube is inserted into the patient's body and the second section of the tube is external to the patient's body. The system can further comprise a dressing configured between an external surface of a patient's body and the flange. The dressing further comprises at least one of: one or more adhesive layers, one or more sealing layers, and one or more gauze layers. The dressing and the flange can be pre-attached to the second section of the tube. In an embodiment the system further comprises a locking member associated with the flange and configured to secure the flange at a location around the second section of the tube. In an embodiment a conduit can be formed in the tube, the conduit being fluidically connected to the inflatable sac and the conduit being fluidically connected to the inflation valve.

### BRIEF DESCRIPTION OF THE FIGURES

The accompanying figures, in which like reference numerals refer to identical or functionally-similar elements throughout the separate views and which are incorporated in and form a part of the specification, further illustrate the embodiments and, together with the detailed description, serve to explain the embodiments disclosed herein.

FIG. 1 depicts a chest tube system and/or apparatus, in accordance with the disclosed embodiments;

FIG. 2 depicts a chest tube system and/or apparatus deployed in a patient, in accordance with the disclosed embodiments;

FIG. 3 depicts an alternative embodiment of a chest tube system and/or apparatus, in accordance with the disclosed embodiments;

FIG. 4 depicts a plan view of a chest tube system and/or apparatus, in accordance with the disclosed embodiments;

FIG. 5 depicts an embodiment of a flange associated with a chest tube system and/or apparatus, in accordance with the disclosed embodiments;

FIG. 6A depicts an embodiment of a flange associated with a chest tube system and/or apparatus, in accordance with the disclosed embodiments;

FIG. 6B depicts another view of a flange associated with a chest tube system and/or apparatus, in accordance with the disclosed embodiments;

FIG. 6C depicts teeth on a platform associated with a chest tube system and/or apparatus, in accordance with the disclosed embodiments;

FIG. 7A depicts a zip tie system associated with a chest tube system and/or apparatus, in accordance with disclosed embodiments;

FIG. 7B depicts another view of a zip tie system associated with a chest tube system and/or apparatus, in accordance with disclosed embodiments;

FIG. 8A depicts an embodiment of a flange associated with a chest tube system and/or apparatus, in accordance with the disclosed embodiments;

FIG. 8B depicts another view of an embodiment of a flange associated with a chest tube system and/or apparatus, in accordance with the disclosed embodiments;

FIG. 9A depicts an embodiment of a flange associated with a chest tube system and/or apparatus, in accordance with the disclosed embodiments;

FIG. 9B depicts another view of an embodiment of a flange associated with a chest tube system and/or apparatus, in accordance with the disclosed embodiments;

FIG. 9C depicts another view of an embodiment of a flange associated with a chest tube system and/or apparatus, in accordance with the disclosed embodiments;

FIG. 10 depicts an embodiment of a flange associated with a chest tube system and/or apparatus in accordance with the disclosed embodiments;

FIG. 11A depicts an embodiment of a flange associated with a chest tube system and/or apparatus, in accordance with the disclosed embodiments;

FIG. 11B depicts components of a flange associated with a chest tube system and/or apparatus, in accordance with the disclosed embodiments; and

FIG. 12 depicts a flow chart of steps associated with a method for performing a thoracostomy using the systems and apparatuses disclosed herein, in accordance with the disclosed embodiments.

### DETAILED DESCRIPTION

The particular values and configurations discussed in the following non-limiting examples can be varied, and are cited merely to illustrate one or more embodiments and are not intended to limit the scope thereof.

Example embodiments will now be described more fully hereinafter with reference to the accompanying drawings, in which illustrative embodiments are shown. The embodiments disclosed herein can be embodied in many different forms and should not be construed as limited to the embodiments set forth herein; rather, these embodiments are provided so that this disclosure will be thorough and complete, and will fully convey the scope of the embodiments to those skilled in the art. Like numbers refer to like elements throughout.

The terminology used herein is for the purpose of describing particular embodiments only and is not intended to be limiting. As used herein, the singular forms “a”, “an”, and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, Aft AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, Aft BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

The embodiments disclosed herein provide a chest tube apparatus that can be used to treat patients who require a chest tube thoracostomy. The embodiments disclosed herein do not require sutures. In the embodiments, a chest tube includes an inflatable sac or balloon (also known as a cuff) and a novel tube-securing flange, which, in conjunction, can be used to secure the chest tube in the patient at the desired position, without sutures.

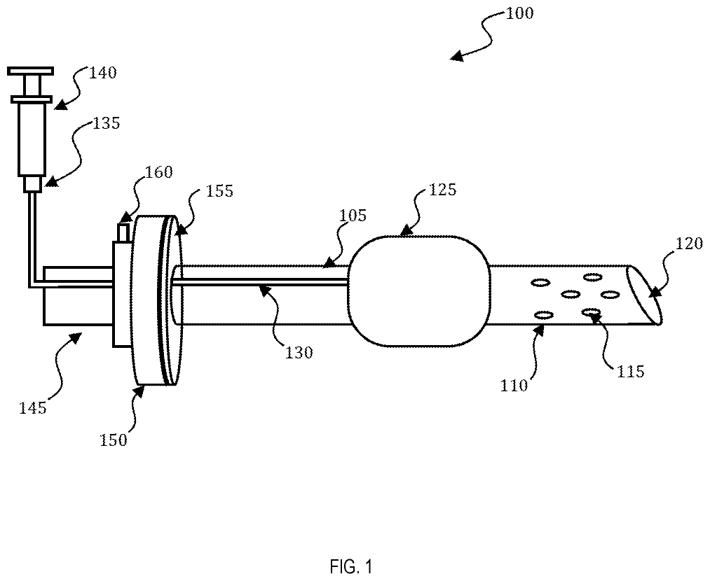

FIG. 1 illustrates an exemplary embodiment of a chest tube system 100. The chest tube system generally includes a chest tube 105 that has a fluted distal (internal to the patient) end 110. The chest tube 105 can be formed of medical grade plastic, rubber, or other such material. It should be appreciated that the chest tube can be sterile, or sterilized, prior to use.

The flutes 115 on the fluted end of the chest tube 110 comprise a series of one or more openings in the chest tube 100. The flutes 115 provide an entry point for liquid or gas to enter the chest tube 105. The flutes 115 are generally ovular in shape, but can be other shapes according to design considerations.

The distal end 110 can further include a shaped opening 120. In an embodiment, the shaped opening 120 can be selected to facilitate insertion of the chest tube into a chest cavity. In particular, the shape can be selected to facilitate insertion of the tube through the incision in the patient, and in and through the tissues between the patient's ribs. As such, in an embodiment, the shaped opening 120 can be pointed, blunt, beveled, conical, sprotte, diamond (Franseen), tuohy, or other such shape that may facilitate insertion of the tube into the chest cavity.

The fluted end of the chest tube 110 further includes an inflatable and deflatable cuff or balloon 125. The balloon 125 is connected to one or more lumens 130 that can be connected to a port or valve 135. The lumen 130 is preferably integrated in the tube 105 or is configured inside tube 105, in order to prevent the lumen from accidentally being pulled into the chest cavity. In other embodiments, the lumen can be formed on the exterior of, or be external from, the tube 105. The lumen 130 allows gas (i.e., air), or medical grade liquid, to flow into, or out of, cuff 125.

The valve 135 is generally configured to allow air (or liquid) to flow into, and out of, the balloon 125 via the lumen 130. The valve 135 can comprise a standard port configured to connect with a syringe, or other source of flowing gas 140. It should be understood that the volume of the cuff 125 can be known, and the volume of gas provided to the cuff, for example, via syringe, can match the maximum cuff volume, in order to avoid over inflation.

The other (external to the patient) end 145 of the chest tube 105 is configured with a flange 150. The flange 150 has a conduit that fits around the chest tube 105, so that the location of the flange 150 on the chest tube 105 can easily be adjusted. The flange 150 further includes a locking mechanism 160 that secures the flange 150 to the chest tube 105 in the desired location. The locking mechanism 160 can comprise any number of arrangements as further disclosed herein. In some embodiments, the flange 150 can be stiff (i.e., formed of hard rubber, plastic, metal, etc.) when a stiff and robust position of the flange is desirable. In other embodiments, the flange 150 may be flexible in order to help provide an airtight seal around an incision.

In additional embodiments, one or more integrated dressings 155 can be configured on the flange and/or on the tube 105. The integrated dressing 155 can comprise one or more of a dry gauze pad, a lubricated pad, an adhesive pad, an additional or integrated Tegaderm™ pad, and the like. It should be understood that one or more of the dressings can be sterile. The integrated dressings 155 can also be provided individually when one of the layers of dressing is not required. In some embodiments, the integrated dressing 155 can have a surface area that far exceeds the size of the incision (e.g., approximately 30 square inches or less) in order to improve the seal provided by the system.

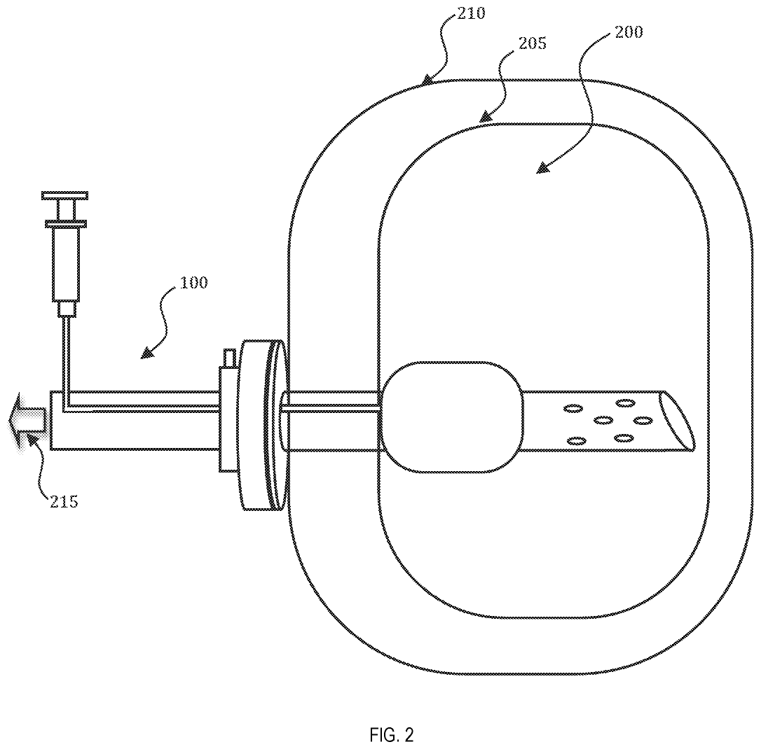

The chest tube system 100 provides an improved means for inserting and securing a chest tube in a patient. FIG. 2 illustrates a view of the chest tube 100 in use. In practice, the distal end 110 (i.e., the end internal to the patient) of tube 105 can be inserted into the chest cavity 200 of a patient. Before insertion, cuff 125 is deflated to improve the ease with which the tube 105 can be inserted.

Once the chest tube is inserted, the cuff 125 can be inflated with air, another gas, or a safe liquid solution, via syringe 140. After insertion, the tube 105 can be retracted slightly so that the cuff 125 fits snuggly against the inner surface 205 of the chest cavity 200. The distal end 110 of the tube 105 is thus secured within the chest cavity 200 and cannot be pulled out.

The flange 150 can be secured on the outside surface 210 of the body. In certain embodiments, the integrated dressings 155 are configured between the incision on the patient and the flange 150. The flange 150 can be secured to the chest tube 105 by locking mechanism 160. The patient's body is thus sandwiched between the cuff 125 on the inside and the flange 150 on the outside, so that the tube 105 is held securely in place. The tube 105 then functions as any standard chest tube allowing liquids and gasses trapped in the chest cavity to escape, as vacuum 215 is pulled through the tube 105. In some embodiments, the tube 105 can be attached to a standard Pleurovac™, or other such vacuum seal device.

Not only is this procedure much faster than prior art approaches, if the location of the chest tube requires adjustment, or removal, it is a simple exercise to release locking mechanism 160, deflate cuff 125, and reposition or remove the chest tube system 100.

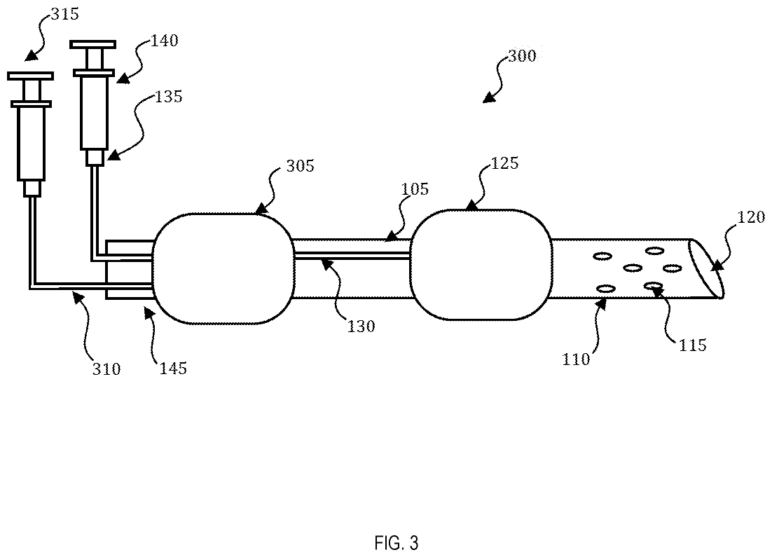

FIG. 3 illustrates an alternative embodiment of a system 300. It should be appreciated that like features are identified with the same reference numerals throughout the figures. In system 300, the flange is replaced by an adjustable exterior balloon 305. In such embodiments, the balloon 305 can be adjusted on the exterior of the patient to secure the tube 105, in the same manner as the flange. When the tube is in its proper position, the balloon 305 can be inflated. The pressure from the inflation of balloon 305 secures the balloon at a desired location along tube 105 so that the tube is secured in place once it is installed in the patient. It should be understood that the adjustable exterior balloon 305 can be integrally connected to lumen 130, or an additional dedicated lumen 310 and valve assembly 315 can be connected to balloon 305 according to design considerations.

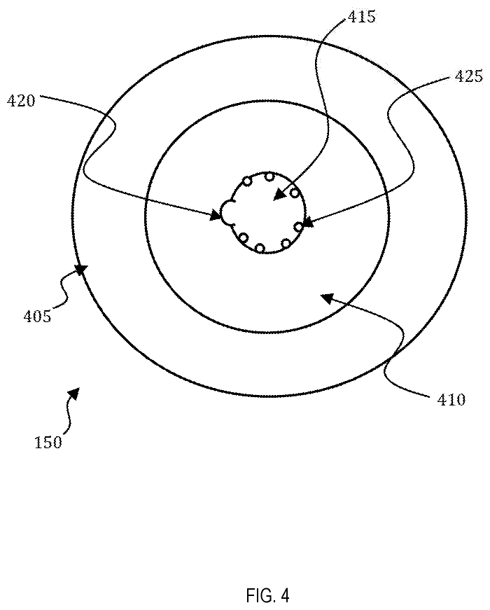

Flange 150 and locking mechanism 160 can be embodied in a number of different ways. FIGS. 4-11 illustrate a variety of embodiments of flange 150 and locking mechanisms 160 that can be implemented with chest tube system 100.

FIG. 4 illustrates an embodiment of flange 150 employing a resistance fit. In general, flange 150 includes a first platform 405, which has a wider diameter, and a second platform 410 that extends from the surface of the first platform 405 in the direction opposite the patient. A conduit 415 for the chest tube 105 passes through both first platform 405 and second platform 410. The conduit can include recess 420 to accommodate lumen 130, when the lumen is exterior to chest tube 105, or when the shape of chest tube 105 is modified by the integration of lumen 130.

In the embodiment illustrated in FIG. 4, a series of resistive protrusions 425, including but not limited to, fluting, dimples, ridges or the like, can be formed on the surface of conduit 415. The resistive protrusions serve to hold the chest tube 105 in place by exerting resistive frictional forces on the chest tube 105. In some embodiments, it may be necessary to squeeze chest tube 105 so that it can pass through conduit 415. Once the chest tube 105 and flange 150 are correctly positioned, the chest tube 105 can be released so that the resistive protrusions hold the chest tube 105 and flange 150 in place.

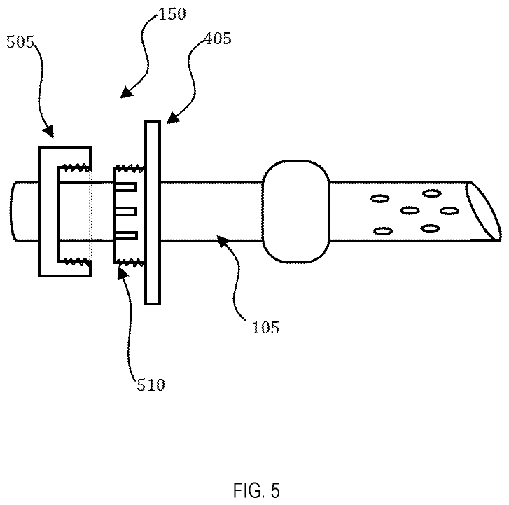

FIG. 5 illustrates an embodiment of flange 150 employing a compression and nut fitting. In this embodiment, the flange 150 comprises a first platform 405 with a male threaded compression sleeve 510 formed thereon. The male threaded compression sleeve is generally configured to fit around chest tube 105, such that when a female compression nut 505 is engaged with the male threaded compression sleeve 510, the sleeve 510 is compressed around the chest tube 105 so that the flange 150 is held in a desired location. It should be appreciated that, in this arrangement and the remaining arrangements, recess 420 may still be included in the flange 150 to accommodate lumen 130.

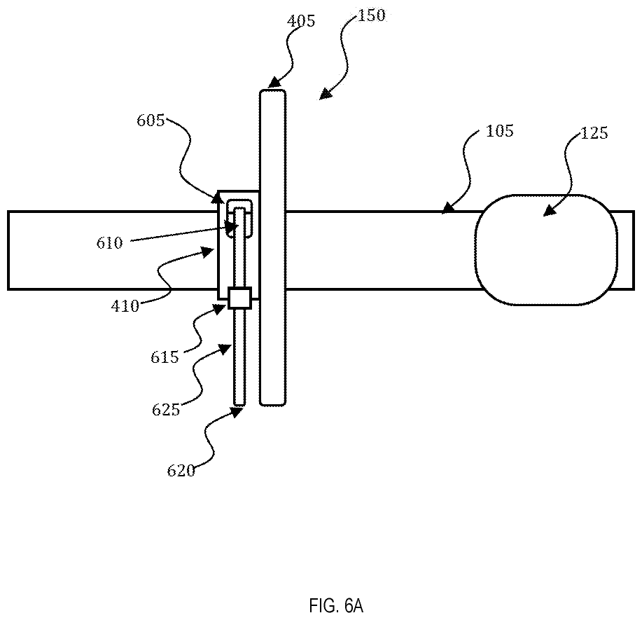

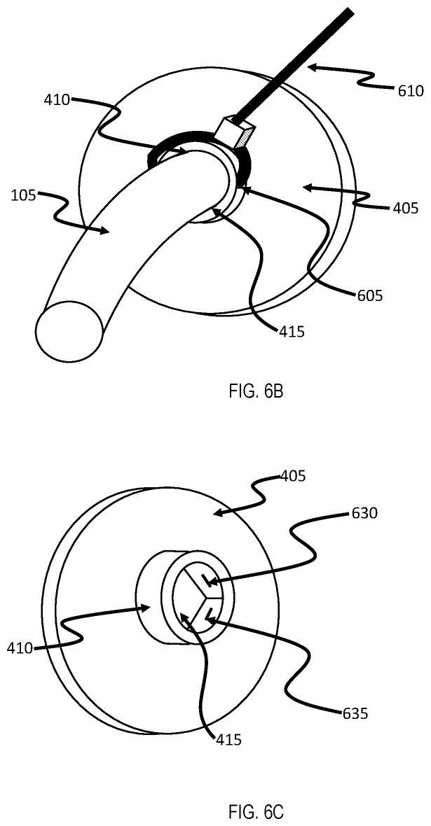

FIG. 6A illustrates an embodiment of flange 150 employing a zip tie retention configuration. In this embodiment, the flange 150 includes first platform 405 and second platform 410, and is configured so that chest tube 105 can pass through conduit 415. The second platform 410 can have two or more slots 605 (only one slot 605 is visible in the figure, but more can be included) through which a zip tie 610 can pass. The slots 605 are preferably biased toward the same side of second platform 410 such that a zip tie 610 (or other such device including string, suture, tape, rubber band, etc.) can be passed through the two slots 605. The chest tube 105 can then be inserted through the conduit 415 and the flange 150 and can be adjusted to the desired position with respect to the chest tube 105. It should be understood that, in other embodiments, the chest tube 105 can be inserted in the conduit 415 before the zip tie 610 is inserted through the slots 605. With the flange 150 properly in place, the zip tie 610 can be secured around a portion of the tube 105 and a portion of the exterior of second platform 410, in order to secure the flange 150 at the desired location.

The zip tie 610 can be made of nylon or other such flexible material. The zip tie has a flexible section 625 with teeth that engage with a pawl 615 at the end that provides a ratchet type lock. As the free end 620 of the flexible section 625 is pulled, the zip tie tightens and the teeth engage in the pawl 615 so that the free end cannot be retracted. In some embodiments, zip ties can include a tab that can be depressed to release the ratchet so that the zip tie can be loosened or removed.

FIG. 6B illustrates a second view of the implementation of the zip tie retention embodiment. Note that the zip tie 610 is preferably secured against the chest tube 105 via slots 605. The zip tie 610 is then secured around the opposite exterior side of second platform 410 in order to provide a strong grip against the chest tube 105 and prevent movement of the flange 150 along the chest tube 105. FIG. 6C illustrates that, in certain embodiments, one or more teeth 630 and 635 can be configured on the internal surface of the second platform 410. In general, when the zip tie 610 is tightened, the teeth 630 and 635 engage against the tube 105 as the tube is pulled tightly against the sides of conduit 415. The teeth 630 and 635 can provide additional grip against tube 105 after it is passed through conduit 415, in order to better secure the tube 105 in place.

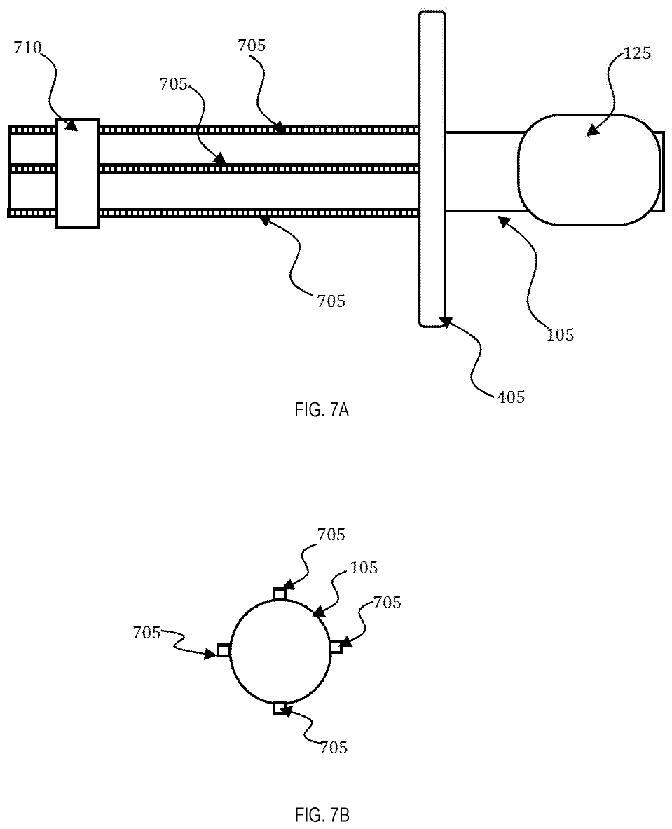

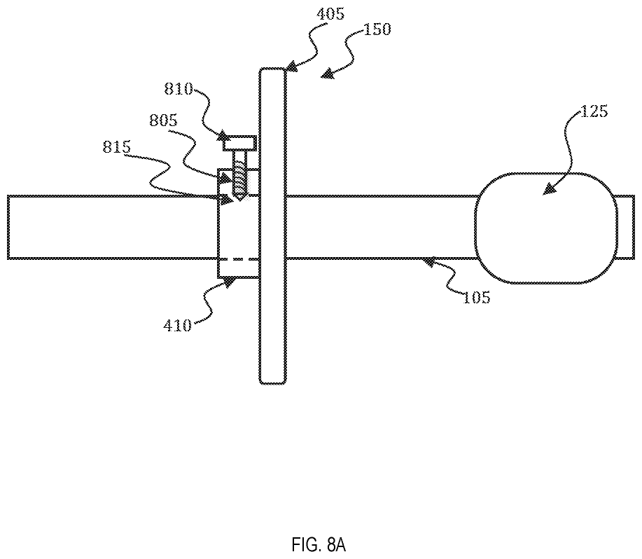

FIG. 7A illustrates an alternative embodiment wherein a zip tie strip of teeth is formed on one or more sides of the chest tube. In this embodiment, the chest tube 105 can be configured with external strips of zip tie teeth 705. The strips 705 can be located at 90 degrees from one another (or at other desired intervals) along the exterior of the chest tube. FIG. 7B illustrates an elevation view of chest tube 105 configured with strips of zip tie teeth 705.

A zip tie nut 710 can be configured to engage the teeth in the strips 705. When the flange 150 is in a desired location, the zip tie nut 710 can be secured against the first platform 405. In cases where it is desirable to adjust the location of the flange 150, the zip tie nut 710 can be rotated (for example, 45 degrees, although other rotational angles may also be applied) so that it disengages from the strips 705. The flange can then be readjusted along the chest tube 105 and the zip tie nut can be reengaged.

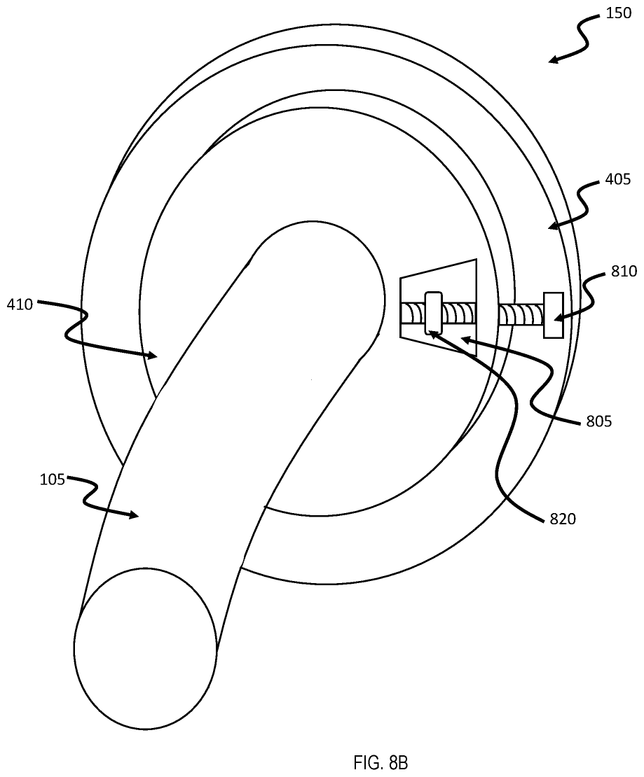

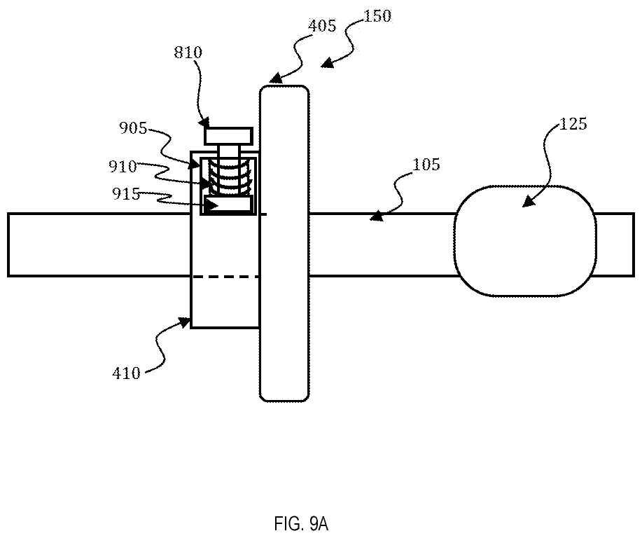

FIG. 8A illustrates another embodiment of flange 150 employing a setscrew fitting to secure the flange 150 in place on chest tube 105. In this embodiment, the flange 150 includes a first platform 405 and second platform 410. The second platform 410 includes a threaded channel 805. The threaded channel 805 is formed to accept a setscrew 810.

When the flange 150 has been adjusted to the correct position on chest tube 105, setscrew 810 is engaged until it is in secure contact with chest tube 105. In some embodiments, setscrew 810 can include a sharp, pointed, jagged, or otherwise engaging tip 815. The tip 815 is intended to form a solid connection with the chest tube 105 so that the flange 150 is held securely in place on the chest tube 105. It should be appreciated that the tip 815 is preferably formed to bite into the chest tube 105 without penetrating into the interior of the chest tube 105.

FIG. 8B illustrates a different view of the setscrew fitting. As illustrated, in some embodiments, a setscrew nut 820 can be provided in channel 805. In such embodiments, the setscrew 810 can be engaged with setscrew nut 820, which holds the screw in place against the chest tube 105.

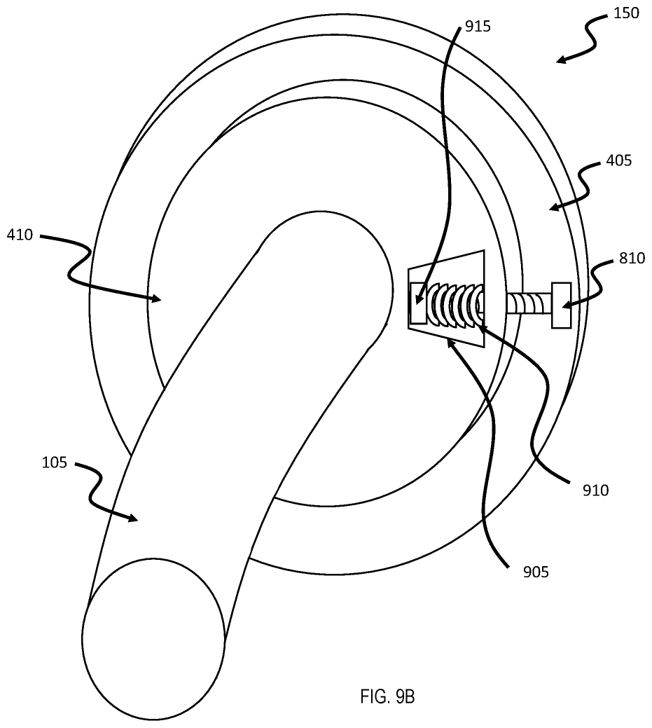

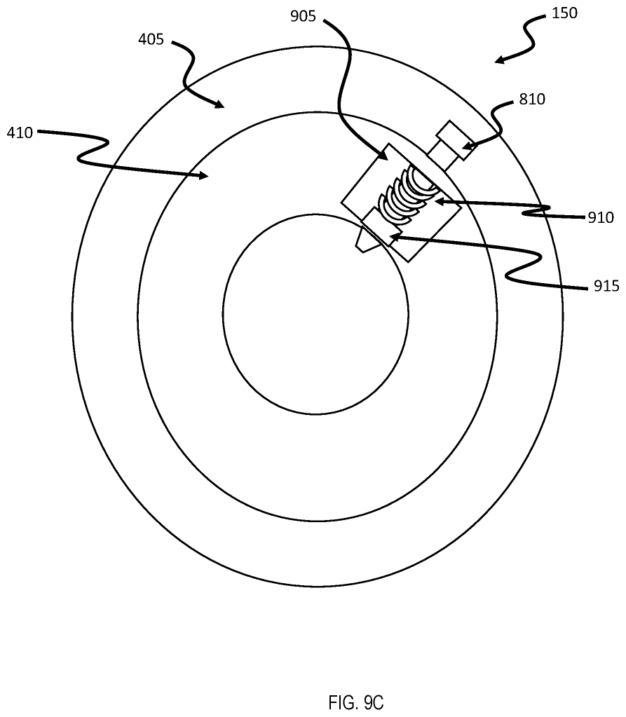

FIG. 9A illustrates an embodiment of flange 150 employing a spring-loaded setscrew fitting. In this embodiment, the flange 150 includes a first platform 405 and second platform 410. The second platform 410 includes a channel 905. The channel 905 is formed to accept a setscrew 810, which is held in place with spring 910.

When the flange 150 has been adjusted to the correct position on chest tube 105, setscrew 810 is engaged until it is in secure contact with chest tube 105. Spring 910 holds the setscrew against the chest tube 105. In some embodiments, setscrew 810 can include a sharp, pointed, or jagged, or otherwise engaging tip 815. The tip 815 is intended to form a solid connection to the chest tube 105 so that the flange 150 is held securely in place on the chest tube 105. It should be appreciated that the tip 815 is preferably formed to bite into the chest tube 105 without penetrating into the interior of the chest tube 105.

FIG. 9B and FIG. 9C illustrate two respective views of the spring-loaded setscrew fitting. As illustrated, in some embodiments, the spring 910, compressed by nut 915, can be provided in channel 905. In such embodiments, the setscrew 810 can be held in place by spring 910 against the chest tube 105.

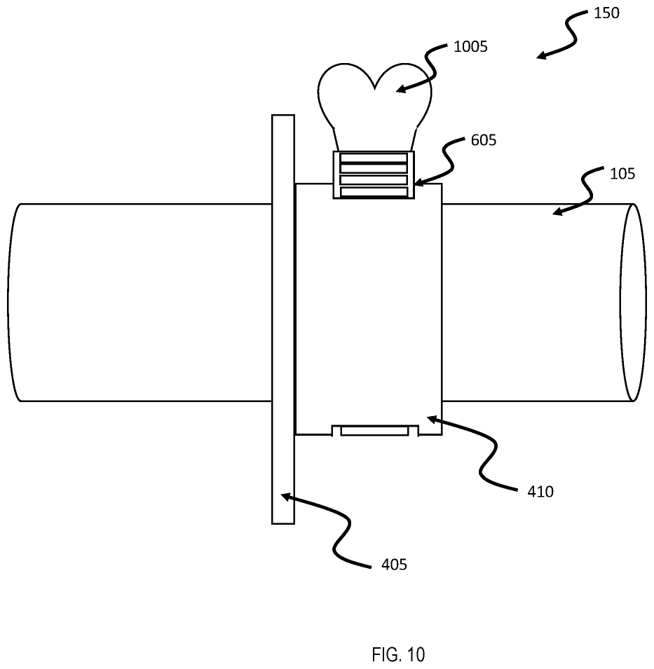

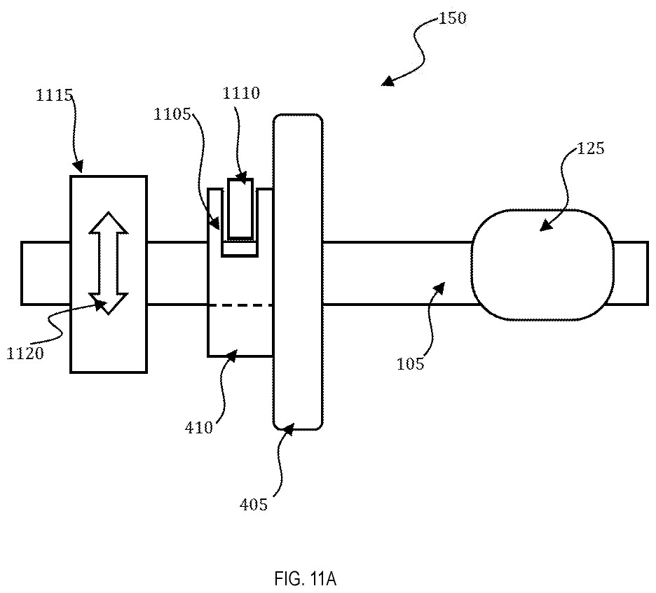

FIG. 10 illustrates an embodiment of flange 150 employing a hose clamp fitting. In this embodiment, the flange 150 includes first platform 405 and second platform 410, and is configured so that chest tube 105 can pass through conduit 415. The second platform has two or more slots 605 through which a clamp 1005 can pass. The slots 605 are preferably biased toward the same side of second platform 410 such that clamp 1005 (or other such device, including a hose clamp, butterfly clamp, etc.) can be passed through the two slots 605. The chest tube 105 can then be inserted through the conduit 415 and the flange 150 can be adjusted to the desired position with respect to the chest tube 105. It should be understood that in other embodiments, the chest tube 105 can be inserted in the conduit 415 before the clamp 1005 is inserted through the slots 605. With the flange 150 properly in place, the clamp 1005 can be secure around the exterior of second platform 410 in order to secure the flange 150 at the desired location.

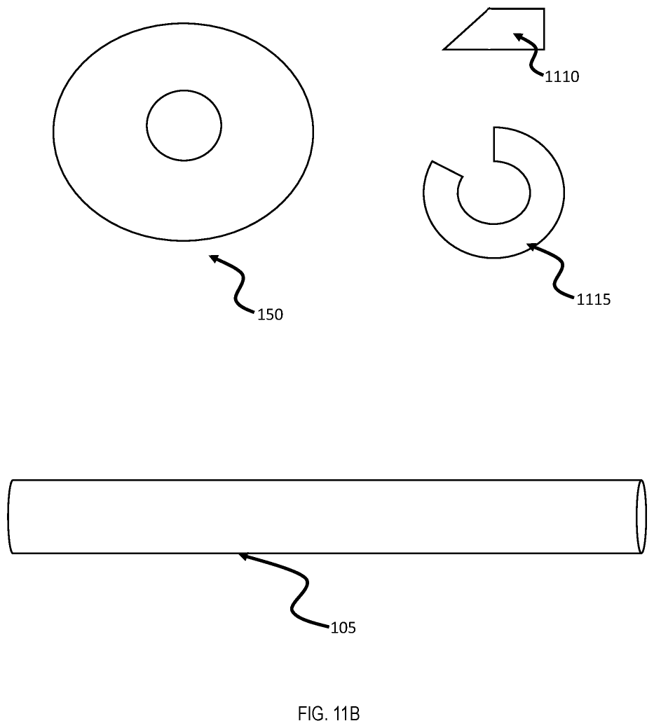

FIG. 11A illustrates an embodiment of flange 150 employing a compression fitting. In this embodiment, the flange 150 includes first platform 405 and second platform 410, and is configured so that chest tube 105 can pass through conduit 415. The second platform 410 includes an orifice 1105 and a retention plug 1110. The retention plug 1110 is formed to fit in the orifice 1105. The embodiment further includes a clamping cap 1115 that comprises a semicircular or crescent shaped cap. The interior diameter of the clamping cap matches, or is slightly larger than, the diameter of second platform 410. The various components of this embodiment are illustrated in FIG. 11B.

The chest tube 105 can be inserted through the conduit 415 and the flange 150 can be adjusted to the desired position with respect to the chest tube 105. With the flange 150 properly in place, the clamping cap 1115 can be slid around the exterior of second platform 410. The opened portion of the clamping cap 1115 is positioned so that the orifice 1105, and retention plug 1110, remains exposed.

In order to secure the flange 150 at the desired location, the clamping cap 1115 can be rotated as illustrated by arrow 1120, such that the retention plug 1110 is forced into contact with the chest tube 105. In certain embodiments, the retention plug 1110 can have a textured or gripping bottom surface so that additional friction is present between the retention plug 1110 and the chest tube 105, when the clamping cap 1115 is rotated into place. In order to release the flange 150, the clamping cap is rotated, again according to arrow 1120, until the gap in the clamping cap 1115 exposes the retention plug 1110.

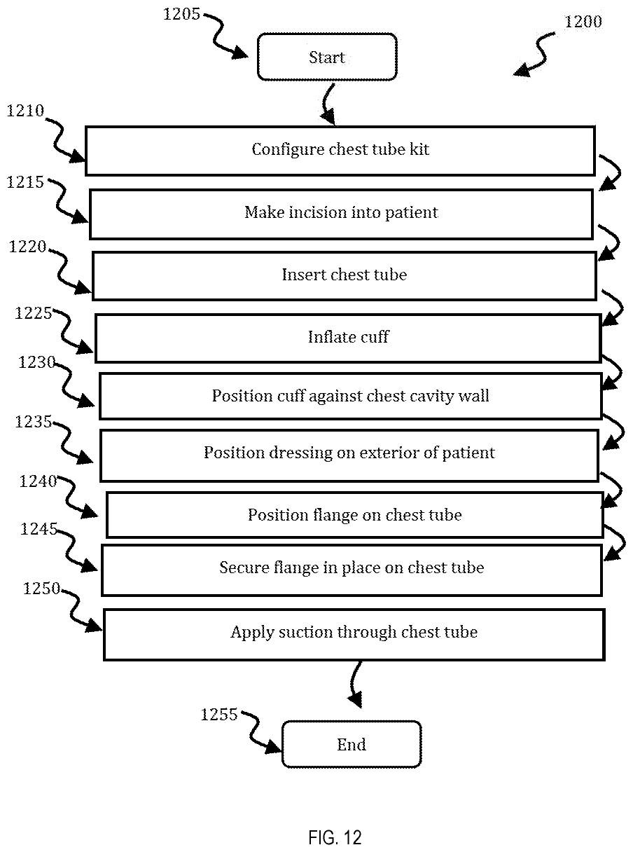

FIG. 12 illustrates steps of a method for securing a chest tube in a patient according to the embodiments disclosed herein. The method begins at step 1205. At step 1210, a chest tube device can be configured according to the embodiments presented herein. The device can be comprised of a kit including a chest tube with an integrated cuff and lumen, a flange, a knife or scalpel, and one or more clamps including a mayo clamp, perfusionist clamp, or other similar clamps. The entire kit can be sterilized.

Next, at step 1215, an incision can be made in the patient's chest area. At step 1220, the chest tube can be inserted into the patient's chest. It should be appreciated that the chest tube can be inserted such that it does not pinch or damage the neurovascular bundle that runs along the patient's ribs. The chest tube is inserted far enough that the balloon or cuff, integrated on the tube, is positioned inside the patient's chest cavity. Preferably, the tube is not inserted too far into the chest cavity, in order to avoid tube blockage, and to avoid damage to the heart or blood vessels in the chest.

At step 1225, the balloon or cuff inside the chest cavity can be inflated, and the chest tube can be positioned so that the cuff is snuggly positioned against the interior wall of the chest cavity at step 1230.

Next at step 1235, a dressing can be positioned against the exterior of the patient. It should be appreciated that in some embodiments, the dressing can be one or more layers of dressing integrated on the first platform of the flange. In other embodiments, the dressing can be a standalone arrangement of one or more layers of dressing with a hole that allows the dressing to fit around the chest tube. In either case, the dressing can include sterilized gauze layers (regular or Vaseline™ gauze), sterilized adhesive layers, and can be soft or rigid. In some embodiments, one or more of the layers can be Tegaderm™ or other such material.

At step 1240, the flange can be positioned along the chest tube such that the chest tube is held in a desired location. In most cases, the flange can be snugged up against the exterior of the patient, effectively sealing the incision in the patient. The inflated balloon serves to hold the chest tube in place inside the chest cavity, while the flange holds the chest tube in place on the exterior of the chest cavity. At step 1245, the flange can be secured in place using any of the retention mechanism embodiments disclosed herein.

With the chest tube securely in place, suction can be applied through the chest tube in order to draw fluid or gas out of the chest cavity as shown at step 1250. In certain embodiments, this can be accomplished with a Pleurovac™ device or other similar device. The method ends at step 1255.

It should be appreciated that in cases where a patient is suffering from a pneumothorax (i.e., a simple or tension pneumothorax), a hemothorax (again including a simple or tension hemothorax), or other such condition, it is often critically important to insert a chest tube as quickly as possible to relieve the pressure building in the chest cavity. The systems and methods disclosed herein provide an extremely efficient means for inserting a chest tube and sealing the incision in the patient; no sutures are required. Also, the systems and methods disclosed herein prevent unsterile regions of the tube from entering the body. The disclosed embodiments are thus faster and more effective than prior art methods for securing a chest tube in place, which can be critical in emergency trauma situation.

Based on the foregoing, it can be appreciated that a number of embodiments, preferred and alternative, are disclosed herein. For example, in an embodiment, a system comprises a tube for insertion in a patient's chest cavity, an inflatable sac configured on a first section of the tube, an inflation valve for inflating the inflatable sac, and a flange associated with a second section of the tube configured for securing the tube. In an embodiment, the first section of the tube is inserted into the patient's body and the second section of the tube is external to the patient's body.

In an embodiment, the system further comprises a dressing configured between an external surface of a patient's body and the flange. In an embodiment, the dressing further comprises at least one of: one or more adhesive layers, one or more sealing layers, and one or more gauze layers. In an embodiment the dressing and the flange are pre-attached to the second section of the tube.

In an embodiment, the system further comprises a locking member associated with the flange and configured to secure the flange at a location around the second section of the tube.

In an embodiment, the system further comprises a conduit formed in the tube, the conduit being fluidically connected to the inflatable sac and the conduit being fluidically connected to the inflation valve.

In another embodiment, a chest tube apparatus comprises a tube for insertion in a patient's chest cavity, an inflatable cuff configured on the tube, an inflation valve for inflating the inflatable cuff, and a flange associated with the tube configured for securing the tube. In an embodiment, the tube further comprises a first section and a second section, wherein the cuff is formed on the first section of the tube and is inserted into the patient's body, and wherein the second section of the tube holds the flange and is external to the patient's body.

In an embodiment, the apparatus further comprises a dressing configured between an external surface of a patient's body and the flange. The dressing further comprises at least one of: one or more adhesive layers, one or more sealing layers, and one or more gauze layers. The dressing and the flange are pre-attached to the second section of the tube. In an embodiment the dressing is integrated on the flange.

In an embodiment, the apparatus further comprises a locking member associated with the flange and configured to secure the flange at a location around the tube.

In an embodiment, the apparatus further comprises a conduit formed in the tube, the conduit being fluidically connected to the inflatable cuff and the conduit being fluidically connected to the inflation valve.

In yet another embodiment, a method for chest tube thoracostomy comprising: inserting a chest tube in a patient, the chest tube comprising an inflatable cuff, an inflation valve, and a flange, inflating the inflatable cuff via the inflation valve, adjusting a position of the chest tube in the patient, and securing the chest tube at the position with the flange.

In an embodiment, adjusting a position of the chest tube further comprises drawing the chest tube out of the patient until the cuff contacts an internal chest wall of the patient.

In an embodiment the method further comprises applying a dressing between an external surface of a patient's body and the flange. In an embodiment the dressing further comprises at least one of: one or more adhesive layers, one or more sealing layers, and one or more gauze layers.

In an embodiment securing the chest tube at the position with the flange further comprises engaging a locking member associated with the flange to secure the flange at the location around the tube.

It will be appreciated that variations of the above-disclosed and other features and functions, or alternatives thereof, may be desirably combined into many other different systems or applications. Various presently unforeseen or unanticipated alternatives, modifications, variations or improvements therein may be subsequently made by those skilled in the art which are also intended to be encompassed by the following claims.

What is claimed is:

1. A system comprising:
a tube for insertion in a patient's chest cavity;
an inflatable sac configured on a first section of said tube;
an inflation valve for inflating said inflatable sac; and
a flange associated with a second section of said tube configured for securing said tube.

2. The system of claim 1 wherein said first section of said tube is inserted into said patient's body and said second section of said tube is external to said patient's body.

3. The system of claim 1 further comprising:
a dressing configured between an external surface of a patient's body and said flange.

4. The system of claim 3 wherein said dressing further comprises at least one of:
one or more adhesive layers;
one or more sealing layers; and
one or more gauze layers.

5. The system of claim 3 wherein said dressing and said flange are pre-attached to said second section of said tube.

6. The system of claim 1 further comprising:
a locking member associated with said flange and configured to secure said flange at a location around said second section of said tube.

7. The system of claim 1 further comprising:
a conduit formed in said tube, said conduit being fluidically connected to said inflatable sac and said conduit being fluidically connected to said inflation valve.

8. A chest tube apparatus comprising:
a tube for insertion in a patient's chest cavity;
an inflatable cuff configured on said tube;
an inflation valve for inflating said inflatable cuff; and
a flange associated with said tube configured for securing said tube.

9. The apparatus of claim 8 wherein said tube further comprises a first section and a second section, wherein said cuff is formed on said first section of said tube and is inserted into said patient's body, and wherein said second section of said tube holds said flange and is external to said patient's body.

10. The apparatus of claim 8 further comprising:
a dressing configured between an external surface of a patient's body and said flange.

11. The apparatus of claim 10 wherein said dressing further comprises at least one of:
one or more adhesive layers;
one or more sealing layers; and
one or more gauze layers.

12. The apparatus of claim 10 wherein said dressing and said flange are pre-attached to said second section of said tube.

13. The apparatus of claim 10 wherein said dressing is integrated on said flange.

14. The apparatus of claim 8 further comprising:
a locking member associated with said flange and configured to secure said flange at a location around said tube.

15. The apparatus of claim 8 further comprising:
a conduit formed in said tube, said conduit being fluidically connected to said inflatable cuff and said conduit being fluidically connected to said inflation valve.

16. A method for chest tube thoracostomy comprising:
inserting a chest tube in a patient, said chest tube comprising an inflatable cuff, an inflation valve, and a flange;
inflating said inflatable cuff via said inflation valve;
adjusting a position of said chest tube in said patient; and
securing said chest tube at said position with said flange.

17. The method claim 16 further wherein adjusting a position of said chest tube further comprises:
drawing said chest tube out of said patient until said cuff contacts an internal chest wall of said patient.

18. The method of claim 16 further comprising:
applying a dressing between an external surface of a patient's body and said flange.

19. The method of claim 16 wherein said dressing further comprises at least one of:
one or more adhesive layers;
one or more sealing layers; and
one or more gauze layers.

20. The method of claim 16 wherein securing said chest tube at said position with said flange further comprises:
engaging a locking member associated with said flange to secure said flange at said location around said tube.
